(19) United States
(12) Patent Application Publication
(10) Pub. No.: US 2020/0345530 A1
(43) Pub. Date: Nov. 5, 2020

(54) SUPPORT SHELL ARRANGEMENT

(71) Applicant: GELENIC AG, Widnau (CH)

(72) Inventors: Michael NATSIS, Uitikon-Waldegg (CH); Juan GOMEZ DIAZ, Balzers (LI)

(73) Assignee: GELENIC AG, Widnau (CH)

(21) Appl. No.: 16/762,540

(22) PCT Filed: Nov. 8, 2018
(86) PCT No.: PCT/EP2018/080641

§ 371 Date: May 8, 2020

(30) Foreign Application Priority Data
Nov. 9, 2017 (EP) 17200855.9

(51) Int. Cl. A61F 5/01

(52) U.S. Cl. CPC A61F 5/0127; A61F 2005/0137; A61F 2005/0179; A61F 2005/0158

(57) ABSTRACT

A support shell assembly (1) comprises a calf part (2), a foot part (3), and a locking device (5) with a guide rail (52) and with a locking element (51). The locking element (51) is displaceable along the guide rail (52) for setting a bending angle (α) between calf part (2) and foot part (3). The locking device (5) also comprises a spring element (53) which applies a spring force to the locking element (51) in such a way that displacement along the guide rail (52) is made easier or more difficult. An inlay (7) for a support shell assembly comprises a first chamber (71) and a second chamber (72). By means of a first and second valve device (73, 74), a fluid can be filled into the first chamber or discharged from the second chamber to stiffen the inlay (7) in a respective area.

### TECHNICAL FIELD

The present invention relates to a support shell assembly and an inlay, in particular an inner shoe, for a support shell assembly.

### STATE OF THE ART

For the treatment of fractures of the extremities a plaster cast is often not used nowadays. One of the disadvantages of a plaster cast is that the fit of a cast that has hardened on a limb cannot be changed afterwards. If the plaster is too tight, there is a risk of pressure points developing and insufficient blood circulation in the limb.

Especially when treating injuries to joints of the human body or of limbs connected by joints, it is common practice to immobilize the joint and the corresponding limb during the recovery and rehabilitation period by means of a removable support. The state of the art has a large number of different such orthopaedic devices. For example, supports are known which keep the limbs and the joint in a fixed position relative to each other, or those which have a joint which allows bending or freedom of movement. Supports consisting of several shell parts allow flexible adaptation to the limb to be treated. However, a high degree of accuracy in adapting the shell parts to the limb is essential, as the healing process is promoted above all when the support causes the limb to be in an anatomically correct position.

For example, from WO 2009/112164 A1, a support shell assembly with a calf part to accommodate a lower leg and a foot part connected to the calf part via a joint arrangement is known. Furthermore, a bending angle adjustment device is provided on the heel side, by means of which a desired bending angle can be set between the foot part and the calf part, whereby fixing is effected by means of a rotary lock.

The application and especially the exact adjustment of a support, however, require considerable skill and a corresponding effort on the part of the medical staff or the patient.

In order for the supports or support shell assemblies to achieve the necessary stability, they usually have a certain inherent rigidity. However, each patient has a very individual body shape, so that despite flexible support devices, pressure points often occur, which can be uncomfortable and also painful for the patient.

### SUMMARY OF THE INVENTION

Therefore, in a first aspect, it is an object of the present invention to indicate a support shell assembly which allows a flexible and precise adaptation to a limb to be treated and at the same time has a simple handling.

This object is solved with a support shell assembly according to claim 1.

In a second aspect, it is an object of the present invention to indicate an inlay for a support shell assembly which has a high wearing comfort.

This object is solved with an inlay according to claim 15.

According to a first aspect, a support shell assembly is thus indicated which comprises a calf part, a foot part which is pivotally connectable to the calf part, and a locking device with a guide rail attached to the calf part or to the foot part and with a locking element. The locking element is displaceable along the guide rail for adjusting a bending angle between the calf part and the foot part and can be fixed in a desired position on the guide rail. The locking device additionally comprises a spring element which applies a spring force to the locking element in such a way that displacement of the locking element along the guide rail is facilitated or made more difficult.

Due to the articulated connection between the calf part and the foot part, the bending or inclination of the calf part relative to the foot part can be adjusted as required. In other words, the calf part can be pivoted relative to the foot part. The bending angle in this context is understood to be the angle formed between a main longitudinal axis of the calf part and a main longitudinal axis of the foot part and characterizes the extent of the bending or pivoting between the calf part and the foot part. Different bending angles can be set in a range of, for example, 75° to 120°. Possible bending angles are then, for example, 120°, 115°, 100°, 95°, 90°, 85°, 80°, 75°, whereby 90° corresponds to a calf part that is essentially perpendicular to the foot part. A bending angle of 100° means that the calf part has been flexed 10° towards the heel from its vertical position and a bending angle of 60° means that the calf part has been flexed 30° towards the toes from its vertical position. This bending capability allows the support shell assembly to assume an anatomically correct position.

In order to adjust the angle of bending, the support shell assembly has a locking device, the guide rail of which is attached to either the calf part or the foot part and the locking element of which can be moved along the guide rail and fixed in a desired position on the guide rail. Under the desired position, in particular the position which corresponds to the anatomically correct position, i.e. a certain bending angle between the calf part and the foot part, is designated here.

The guide rail may have a latching structure and the locking element may have one or more latching elements, the locking element being movable between a displacement position in which the latching element or elements are out of engagement with the latching structure and a fixing position in which the latching element or elements are in engagement with the latching structure.

In a first embodiment, the spring force of the spring element can have the effect that a displacement of the locking element along the guide rail is facilitated as long as the locking element is not fixed to the guide rail, for example by means of a fixing element. In this way, setting the desired bending angle between the calf part and the foot part can be made easier for the patient or medical staff, since the force required to move the calf part relative to the foot part is then reduced due to the spring element. In addition, the easier movement between the calf part and the foot part caused by the spring element can also allow the patient a certain degree of freedom of movement in a more advanced therapy phase. Due to the articulated connection between the calf part and the foot part, the patient is then allowed to move between the lower leg and foot in a guided manner.

In order to facilitate displacement of the locking element along the guide rail, the spring element in this first embodiment applies a spring force to the locking element in the direction of the displacement position. In doing so, the spring force advantageously effects that the latching element(s) of the locking element are out of engagement with the latching structure of the guide rail.

It is therefore possible in particular to provide a latching connection between the guide rail and the locking element, whereby the application of a force to the locking element by the spring element results in the latching element(s) of the locking element being brought out of engagement with the latching structure of the guide rail. The locking element is then in the displacement position due to the spring force and is held in this position by the spring element. If a force in the opposite direction to the spring force acts on the locking element, the amount of which is greater than the amount of the spring force, the locking element is transferred from the displacement position to the fixing position against the spring force, whereby the latching element(s) of the locking element are brought into engagement with the latching structure of the guide rail. Such an opposing force can be effected in particular by a fixing element.

In an alternative second embodiment, however, the spring force of the spring element can also cause the locking element to be difficult to move along the guide rail after the desired bending angle between the calf part and the foot part has been set, i.e. the locking element and thus also the support shell assembly largely remain in the desired position before the locking element is fixed to the guide rail. This can reduce or even prevent the risk of unintentional displacement of the support shell assembly from the anatomically correct position before the locking element is fixed to the guide rail, thereby ensuring that the support shell assembly is precisely adapted to the limb being treated. The locking element is thus pre-fixed to the guide rail in the desired position by means of the spring element. Since this pre-fixing is effected by the spring force of the locking element, the pre-fixing is to a certain extent automatic. In this case, automatic means that the spring element, without any further action on the part of the user or the use of aids or additional locking structures, ensures that unintentional displacement of the locking element and thus unintentional changes in the deflection angle of the support shell assembly are largely avoided. This ensures easy handling when adjusting the bending angle of the support shell assembly.

In order to make it more difficult to move the locking element along the guide rail with this alternative second embodiment, the locking element is preferably subjected to a spring force in the direction of the fixing position by the spring element. The spring force advantageously effects the above-mentioned automatic pre-fixing.

It is therefore conceivable to provide a latching connection between the guide rail and the locking element, whereby the application of force to the locking element by the spring element results in the latching element(s) of the locking element being brought into engagement with the latching structure of the guide rail. The locking element is then pre-fixed to the guide rail and the locking element is in the fixing position. If a force, the magnitude of which is greater than the magnitude of the spring force, acts on the locking element in the opposite direction to the spring force, the locking element is transferred from the fixing position to the displacement position against the spring force, whereby the engagement between the latching element or elements of the locking element and the latching structure of the guide rail is released. If, for example, a doctor wishes to adjust an anatomically correct position of the support shell assembly, he presses the locking element against the spring force out of engagement with the guide rail and swivels the calf part relative to the foot part or the foot part relative to the calf part until they are at the desired bending angle to each other. If the calf part and the foot part are at a desired bending angle to each other, the physician releases the locking element, whereby the spring force automatically brings the locking element into engagement with the guide rail and pre-fixes the support shell assembly at the desired bending angle.

It is important to understand, however, that a latching connection does not necessarily have to be present with either of the two embodiments mentioned. Instead, it is conceivable that the locking element is only designed in such a way that it can engage behind the guide rail without, however, entering into a latching connection, in the fixing position, for example, a stop is then formed between the areas of the locking element which engage behind the guide rail and the areas of the guide rail which are engaged behind the locking element. Due to the action of the spring element, a static friction is formed between the areas of the locking element and the guide rail which are in contact with each other, which makes it at least more difficult or even impossible to move the locking element along the guide rail. If, in the first embodiment, in which the spring force facilitates the displacement of the locking element, a force opposing the spring force is exerted on the locking element, the locking element is brought into abutment with the guide rail, so that a static friction is formed between the locking element and the guide rail and displacement of the locking element in this correspondingly assumed fixing position is made more difficult. If, in the case of the second embodiment, in which the spring force makes it more difficult to move the locking element, a force opposing the spring force is exerted on the locking element, the static friction between the guide rail and the locking element is reduced or completely eliminated. The locking element is then in the displacement position and can be displaced along the guide rail.

The locking device may further have at least one fixing element, in particular in the form of a fixing screw, for fixing the locking element in the desired position on the guide rail.

This means that an additional fixing of the locking element in the fixing position can be achieved by providing a fixing element which, depending on the embodiment, can exert a fixing force on the locking element in the opposite direction to the spring force or in the same direction. It is advantageous for the fixing element to exert a fixing force on the locking element in the opposite direction to the spring force in the first embodiment and a fixing force in the same direction as the spring force in the second embodiment.

In the first embodiment, the fixing element can, for example, be provided in the form of a fixing screw which can be screwed into a female thread on the locking element in order to bring the locking element into the direction of the fixing position against the spring force caused by the spring element.

In the second embodiment, the locking element can, for example, have a through-opening for a fixing screw, through which the fixing screw is inserted and screwed in the direction of the guide rail until it strikes the guide rail. In doing so, the fixing screw generates a fixing force on the locking element, which applies a force to the locking element in the same way as to the spring element and thus transfers it into the fixing position or fixes it in the fixing position. This fixing force acting in addition to the spring force reduces the risk of unintentional displacement of the calf part along the guide rail in the fixing position, thus further increasing the safety of the support shell assembly with regard to the set deflection angle.

The foot part may be curved on the heel side and may be movable with this curved region at least partially along a correspondingly curved region of the calf part.

For example, the foot part can be inserted with its curved region into the curved region of the calf part, whereby the curved region of the calf part comes to rest on the outside of the support shell assembly. On the other hand, it is also conceivable that the curved region of the calf part is inserted into the curved region of the foot part, whereby the curved region of the foot part comes to lie on the outside of the support shell assembly.

The guide rail of the locking device can be arranged on the heel side of the foot part, in particular formed in one piece with the foot part, or the guide rail of the locking device can be arranged on the heel side of the calf part, in particular formed in one piece with the calf part.

This means that the guide rail may be arranged on the foot part or on the calf part. A embodiments which are made in connection with a guide rail on the foot part therefore apply analogously to a guide rail on the calf part, and vice versa.

The guide rail may be arranged on the foot part and the calf part may have a bulge, depression or recess for receiving the locking element, so that the calf part can be displaced along the guide rail together with the locking element. Alternatively, the guide rail can be arranged on the calf part and the foot part can have a bulge, depression or recess for receiving the locking element, so that the foot part can be displaced along the guide rail together with the locking element.

This bulge, depression or recess or opening is preferably formed in the curved region of the calf part or in the curved region of the foot part and delimits a space in which the locking element can be accommodated with a perfect fit. In the first embodiment, it is preferably a bulge or depression, in the second embodiment preferably a recess.

The guide rail can be arranged on the outside of the foot part and the calf part can have a guide groove on the inside for guided reception of the guide rail.

If the calf part is now placed over the foot part, the guide groove is increasingly guided over the guide rail. In other words, if the foot part is inserted into the calf part, the guide rail is increasingly inserted into the guide groove in the calf part. Similarly, the guide rail can also be arranged on the calf part and inserted in a guided way into a guide groove on the foot part, in both cases, the guide groove ensures that the calf part or foot part cannot tilt sideways.

The locking element can be arranged between the calf part and the foot part, especially in the first embodiment, but also in the second embodiment. Here the locking element is preferably acted upon by the spring element, which can be arranged in particular between the calf part and the locking element or the foot part and the locking element, with a spring force in the direction of the foot part or the calf part.

The calf part may have at least one slit which extends at least partially into the calf part so that a flexible adaptation of the calf part to the calf of a patient is enabled. This slit or several such slits may extend at least partially through the calf part and thereby provide the calf part with a certain deformability or elasticity of form. The slits thus allow the calf part to adapt flexibly to the calf of a patient, while at the same time providing the necessary dimensional stability due to the high inherent rigidity of the calf part.

The support shell assembly may further comprise a sole part which is connectable to the foot part, wherein the sole part has a pressure sensor device for detecting the treading load of the sole part by a patient, and wherein the pressure sensor device comprises at least one pressure sensor which is adapted to output a sensor signal.

The sole part can be detachably connected to the foot part, for example by means of a snap-in connection. Thus, latching projections can be formed on the foot part, which snap in corresponding recesses on the sole part, or vice versa. However, it is also conceivable that the sole part and the foot part are formed in one piece. Preferably, however, a depression corresponding to the outer shape of the sole part is provided in the foot part, into which the sole part can be inserted.

This pressure sensor device can be used to inform the patient about his treading load. In the case of injuries to the leg or foot skeleton or the ligaments and cartilage there, it is particularly important that the patient does not exceed a certain maximum treading load when walking or standing during rehabilitation. As the support shell assembly now has a sole part with one or more pressure sensors, the treading load can be recorded by the pressure sensors and further evaluated.

The support shell assembly can further comprise an evaluation device in which a predetermined maximum value and/or maximum value range of the permissible treading load can be stored and which is adapted to determine a sensor value from the sensor signal of the pressure sensor and to compare the determined sensor value with the predetermined maximum value and/or the maximum value range, and an information transmission unit which is adapted to emit an optical and/or acoustic signal if the determined sensor value exceeds the maximum value and/or the maximum value range. A switch, in particular a push button, may be provided to define a zero point with respect to the treading load. Preferably, the evaluation device is adapted to store this zero point with regard to the evaluation. The zero point can correspond in particular to the load measured by the pressure sensor(s) during normal upright standing.

These elements allow, for example, a physician to program a maximum value or maximum values for an admissible treading load in the evaluation device of the support shell assembly. If the patient then steps on the sole part with a treading load that exceeds these specified maximum values, he is informed of this, for example by means of a flashing warning lamp and/or an audible alarm tone, and can immediately reduce his treading load accordingly. By avoiding overloading, the healing process can be accelerated, which among other things also leads to a reduction in health care costs.

The support shell assembly may further comprise an energy supply device for supplying the evaluation device and/or the information transmission unit with energy, and a work switch, the work switch being adapted to switch on the energy supply in the event of a treading load and to switch off the energy supply if no treading load is detected for a certain time period. By providing energy or power only when the support shell assembly is in use, the lifetime of the energy supply device is extended.

Although the pressure sensor device, the evaluation device, the information transmission unit and the energy supply device have been described here in connection with the support shell assembly, it is equally conceivable to provide these elements in the form of a sole inlay, for example for a plaster or an orthopaedic shoe.

The support shell assembly also advantageously has a tibia part which, together with the calf part, is designed to wrap or surround the lower leg of the patient, preferably completely. Preferably, the tibia part and the calf part are even adapted to surround the lower leg in such a way that they overlap each other in the adjacent areas. Preferably, there is also a foot back part which, together with the foot part, is adapted to wrap or surround, preferably completely wrap or surround, the foot of the patient. The support shell assembly thus exhibits good stability with optimum force flow.

As already mentioned, support shell assemblies must have sufficient stability to provide support, for which purpose their components, such as the foot part or the calf part, have a certain inherent rigidity. Despite adaptable, flexible support devices, unpleasant pressure points for the patient can still occur.

In a second aspect, therefore, an inlay, in particular an inner shoe, is indicated for a support shell assembly, in particular for a support shell assembly as described above, which comprises a first chamber and a second chamber. The inlay also has a first valve device by means of which a fluid such as in particular air can be introduced into the first chamber in order to stiffen the inlay in a first area. The second chamber contains a plurality of moulded bodies. The inlay also has a second valve device by means of which a fluid can be discharged from the second chamber in order to stiffen the inlay in a second area.

A possible application of this inlay can be that it is used in a support shell assembly and thus comes into contact with a patient. Then the still unfilled first chamber is successively filed with a fluid until it lies against the body part to be supported like a cushion, and the second chamber is evacuated, with its moulded bodies lying close to the contours of the body part to be supported. Via the first valve device, the fluid can be discharged from the first chamber at any time, or via the second valve device, a fluid can be let into the second chamber at any time, so that the inlay can be reversibly stiffened. The inlay is thus optimally adapted to the contours of the part of the body to be supported, thus avoiding pressure points and ensuring a high level of comfort of the support shell assembly.

A particularly simple and space-saving embodiment is achieved when the first and second valve devices are formed by a common pumping device which can be switched from a pumping mode to a suction mode and vice versa. The common pumping device preferably has a single bellows, which can be used for both the pumping and the suction function.

However, it should be understood that this inlay can not only be used with a support shell assembly as described above, but can also be inserted into a one-piece support or in a plaster, for example. Nor is its use restricted to the medical field. For example, the inlay can also be used as an inner shoe for a sports shoe such as a ski boot or hiking boot. Conversely, a support shell assembly with the aforementioned locking device does not necessarily have to have such an inlay. Therefore, the aforementioned inlay can be sold and marketed independently of the specified support shell assembly.

The inlay may further comprise a holder in which an air pump can be held, whereby the air pump can be connected to the first valve device for filling the first chamber with air. This holder can, for example, be in the form of a side pocket into which the air pump is inserted and is thus always carried along with the inlay. This enables a patient to adjust the inlay at any time.

### BRIEF DESCRIPTION OF THE DRAWINGS

Preferred embodiments of the invention are described in the following with reference to the drawings, which are for explanatory purposes only and are not to be interpreted restrictively.

In the drawings it is shown:

FIG. 1 a perspective view of a support shell assembly according to a first embodiment according to the invention without tibia and foot back part and without fastening straps, but with sole part, in a first bending position;

FIG. 2 a perspective view of the support shell assembly according to FIG. 1 with tibia and foot back part and with fastening straps and sole part, in a second bending position;

FIG. 3 a perspective partial exploded view of the support shell assembly according to FIG. 1 from diagonally below without tibia and foot back part, without sole part and without fastening straps;

FIG. 4 a detailed view of area A in FIG. 3;

FIG. 5 a perspective view of a support shell assembly according to a second inventive embodiment with sole part and a fastening strap, but without tibia and foot back part;

FIG. 6 a further perspective view of the support shell assembly according to FIG. 5 without fastening strap, tibia and foot back part and sole part;

FIG. 7 a partial exploded view of the support shell assembly as shown in FIG. 5 without fastening strap and tibia and foot back part, but with sole part;

FIG. 8 another partial exploded view of the support shell assembly as shown in FIG. without fastening strap, tibia and foot back part and sole part;

FIG. 9 a detailed view of area B in FIG. 8;

FIG. 10 another partial exploded view of the support shell assembly as shown in FIG. without fastening strap, tibia and foot back part and sole part;

FIG. 11 another partial exploded view of the support shell assembly as shown in FIG. without fastening strap, tibia and foot back part and sole part:

FIG. 12 a perspective view of the support shell assembly as shown in FIG. 5 without fastening strap, tibia and foot back part, but with the sole part not connected;

FIG. 13 a perspective view of the support shell assembly as shown in FIG. 5, with the sole part shown as a partial section;

FIG. 14 a schematic side view of the sole part as shown in FIG. 13;

FIG. 15 a perspective view of an inner shoe with a first and second chamber for a support shell assembly; and

FIG. 16 a schematic sectional view of the second chamber along the plane C-C in FIG. 15 in an air-filled and in an evacuated state.

### DESCRIPTION OF PREFERRED EMBODIMENTS

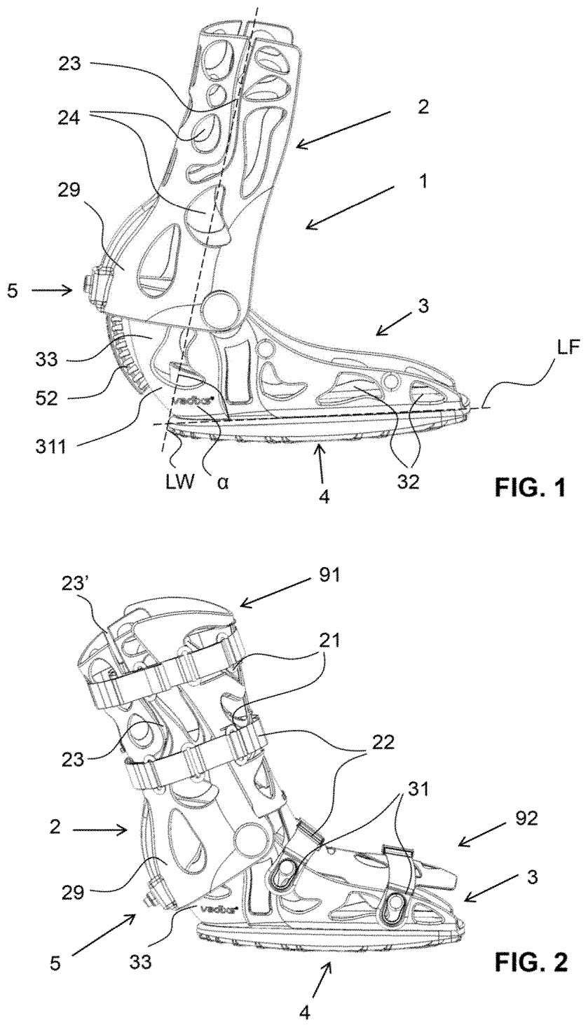

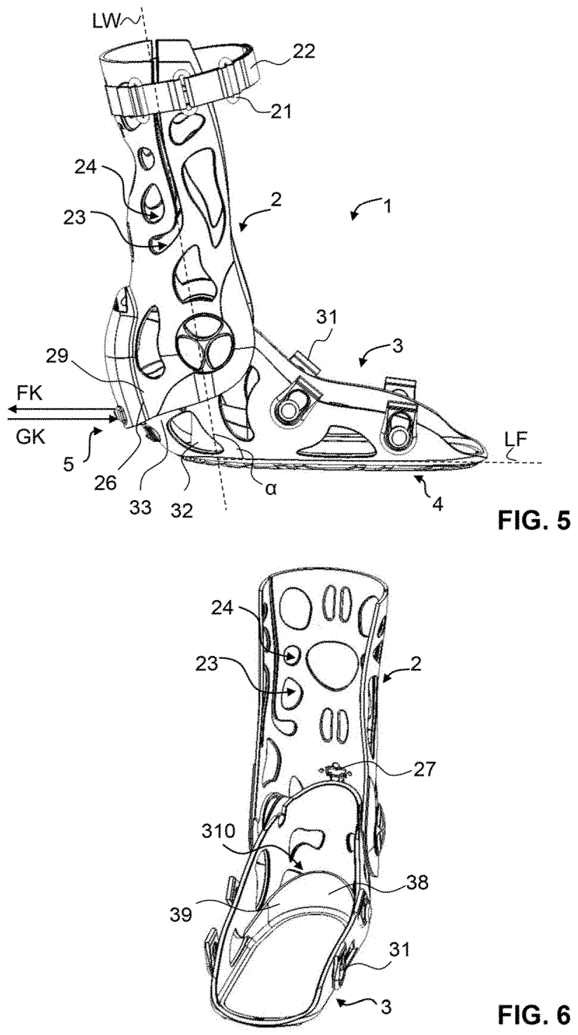

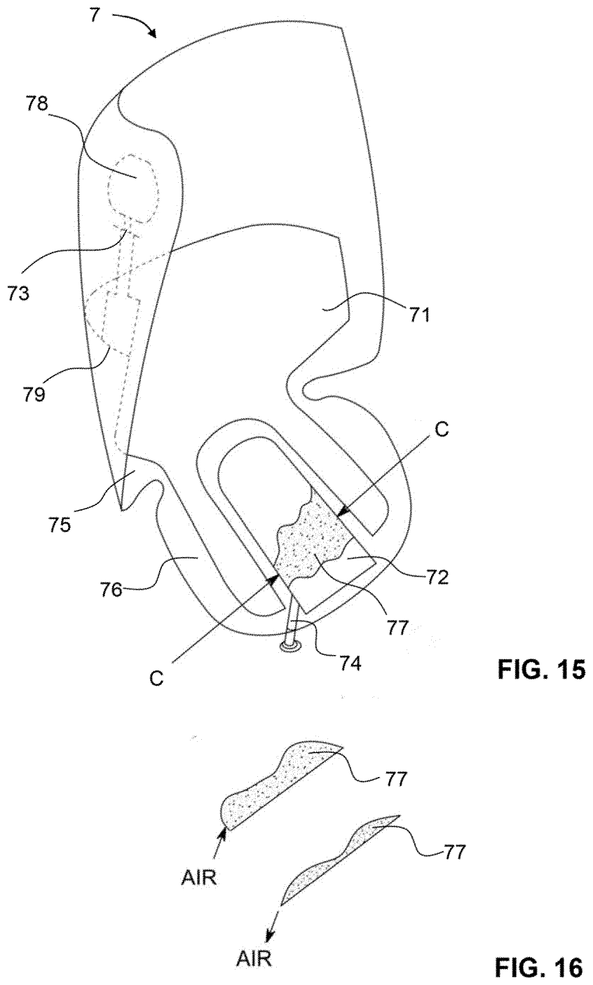

FIGS. 1 to 4 show different views of a support shell assembly 1 according to a first embodiment comprising a calf part 2, a foot part 3, a sole part 4 and a locking device 5. In the support shell assembly 1 according to this first embodiment shown in FIGS. 1 to 4, a spring element 53 serves to facilitate changing the bending angle between the calf part 2 and the foot part 3. A support shell assembly 1 according to a second embodiment is shown in FIGS. 5 to 14, wherein this second embodiment, in contrast to the first embodiment, is has a spring element 53 which serves to make it more difficult to change the bending angle between the calf part 2 and the foot part 3. An inner shoe 7, which can be used as an inlay for a support shell assembly 1 according to the first or also the second embodiment, is shown in FIG. 15.

In the following, in the case of different embodiments, features which have the same or a similar design and/or the same or a similar technical effect are each provided with the same reference signs.

As can be seen from FIGS. 1 to 4, the support shell assembly 1 according to the first embodiment has a calf part 2 for receiving a lower leg and a foot part 3 which can be connected to the calf part 2 at the heel. For the arrangement of the support shell assembly 1 on the lower leg of a patient in a force-fit manner, belt straps 21, 31 are provided on the calf part 2 and on the foot part 3, to which a fastening belt 22 or fastening strap can be fastened in each case. For example, a length-adjustable, so-called Velcro strap can be used, which is passed through the belt straps 21 of the calf part 2 and is looped around the patient's calf and thus fastens the lower leg of a patient to the support shell assembly. A tibia part 91 and a foot back part 92 may be provided between the fastening belts 22 and the lower leg or foot for further stabilization. Together with the calf part 2, the tibia part 91 is adapted to surround or wrap around the patient's lower leg preferably completely. Thanks to this wrapping of the lower leg in combination with the fixation of the foot relative to the lower leg caused by the locking device, an optimal stabilization of the ankle joint is achieved.

The forces occurring during walking are absorbed by the support shell assembly 1 and can be easily dissipated due to the aforementioned wrapping of the lower leg.

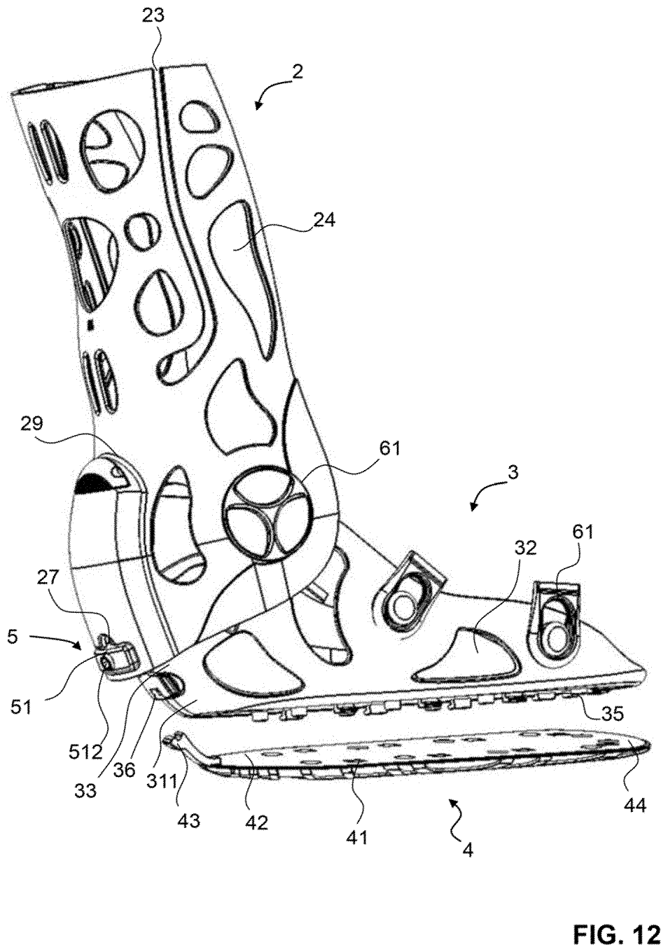

In the present case, the calf part 2 is constructed in one piece and has a slit 23, 23 on both sides, which, seen from the entry side of the calf part 2, extends at least partially into the calf part 2. These slits 23, 23′ give the calf part 2 a certain deformability or elasticity of form and thus enable a flexible adaptation of the calf part 2 to the patient's calf. Both the calf part 2 and the foot part 3 have additional openings 24, 32, which serve a ventilation function. The sole part 4 is discussed in more detail in connection with FIGS. 12 to 14.

In the Figures shown here the foot part 3 is bent on the heel side and is at least partially inserted into the calf part 2 when the calf part 2 is moved in the direction of the foot part 3 with this curved region 311. For this purpose, the calf part 2 also has a correspondingly curved region 29 on the heel side, which is put over the curved foot part when the foot part 3 is pushed into the calf part 2.

Furthermore, a locking device 5 is provided, which comprises a locking element 51, a guide rail 52 and a spring element 53. In the example shown here, the guide rail 52 is formed on the outside in a proximal region 33 of the foot part 3, i.e. on the heel side of the foot part 3. The guide rail 52 extends essentially along the entire curved region 311 of the foot part 3 and slightly beyond the foot part 3 in the direction of the calf part 2.

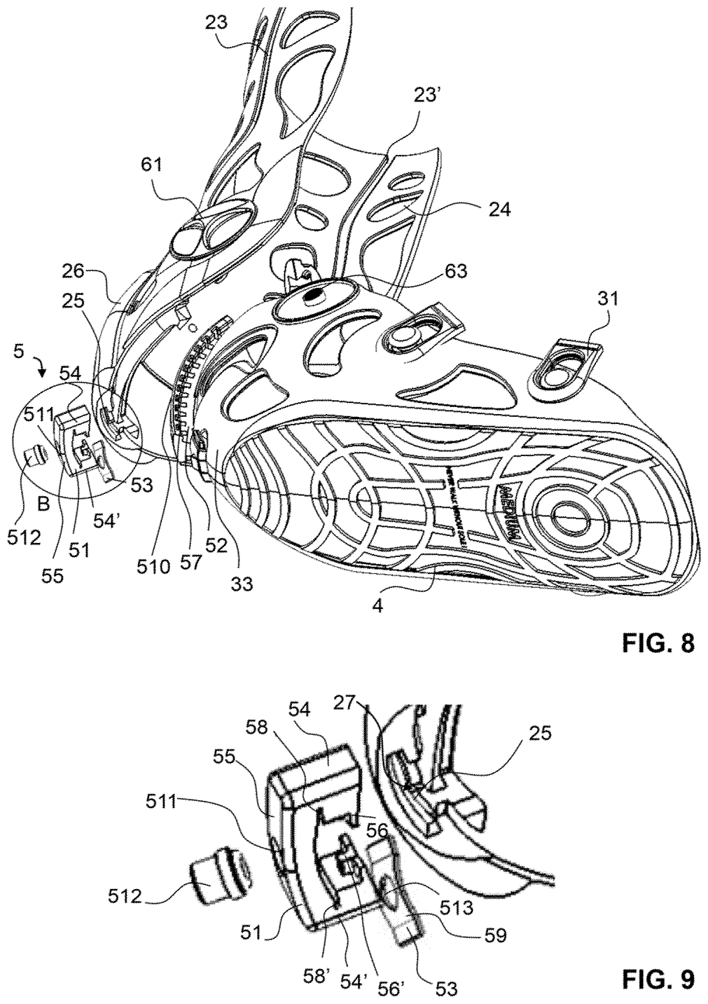

When the calf part 2 is joined to the foot part 3, the guide rail 52 is first accommodated in a guide groove 25, which is advantageously formed on the inside in the curved region 29 of the calf part 2. The guide groove 25 is not visible in the first embodiment shown in FIGS. 1 to 4, but is advantageously present. In relation to the second embodiment, the guide groove 25 is clearly visible in FIGS. 8 and 9. During further assembly, the remaining area of the guide rail 52 is increasingly pushed into the guide groove 25. If the calf part 2 is essentially perpendicular to the foot part 3, the guide rail 52 is practically completely accommodated in the guide groove 25.

As can be seen in FIGS. 8 and 9, the guide groove 25 is designed as a T-shaped recess in which the complementary guide rail 52 is accommodated almost without play in the form of a T-shaped elevation. Thanks to this guided movement, lateral tilting of the calf part 2 in relation to the foot part 3 can be avoided. Depending on the displacement of the calf part 2 along the guide rail 52 of foot part 3, a certain bending angle α is set between a main longitudinal axis LW of the calf part 2 and a main longitudinal axis LF of the foot part 3. In order to fix the calf part 2 in a desired position or at a certain bending angle α, the locking device 5, in particular the locking element 51 with the spring element 53, is provided.

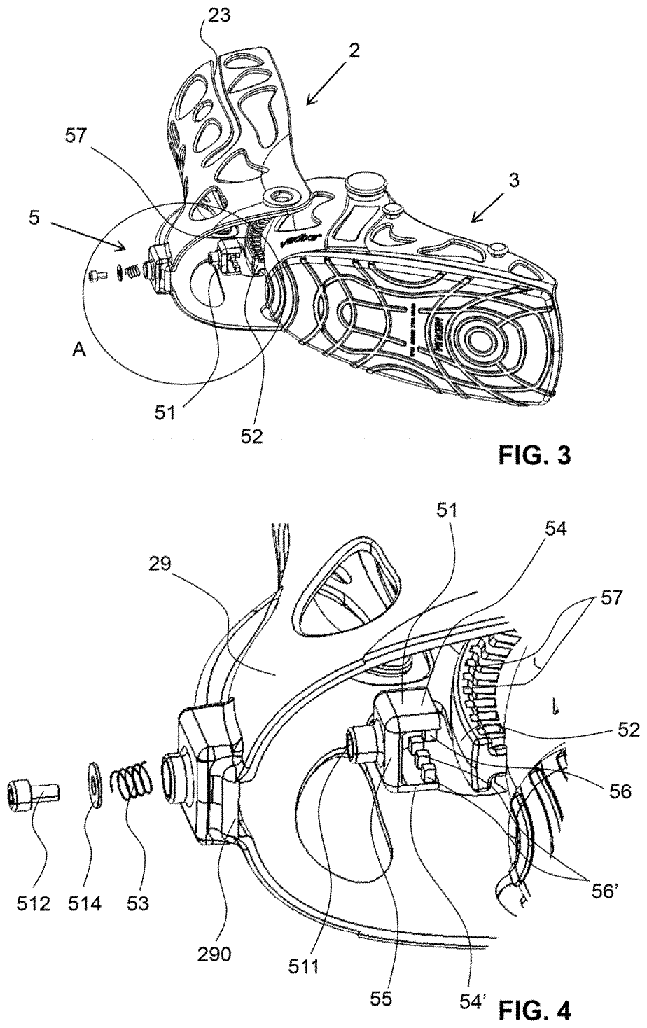

In the curved region 29 of the calf part 2, a bulge 290 is formed extending in the direction away from the foot part 3 and outwards, which serves to receive the locking element 51 in a precisely fitting manner (see FIGS. 3 and 4). The locking element 51 is thus arranged between the calf part 2 and the foot part 3. The locking element 51 is essentially U-shaped as a whole with two legs 54, 54′ and a bar 55 connecting these two legs 54, 54′. A central through-opening 511 is formed in the bar 55, inside which an internal thread is formed. When adjusting the bending angle α between the calf part 2 and the foot part 3, the locking element 51, which is accommodated in the bulge 290, is moved along the guide rail 52 together with the calf part 2.

However, it is equally conceivable that the guide rail 52 is not located on the foot part 3 but on the calf part 2, and that the locking element 51 can be accommodated accordingly in a depression in the proximal region 33 of the foot part 3. All statements relating to a guide rail 52, which is arranged on the foot part 3, or to a locking element 51, which can be accommodated in the calf part 2, therefore also apply analogously to a guide rail which is arranged on the calf part 2 or to a locking element which can be accommodated in the foot part 3. In addition, the locking element 51 could in principle also be connected to the calf part 2 (or the heel part 3) or even be attached to it in one piece. This would be possible, for example, if the curved region 29 of calf part 2 had a certain flexibility that would allow the locking element 51, which is formed in one piece with it, to be moved towards and away from guide rail 52.

As shown further in FIGS. 3 and 4, the two legs 54, 54′ of the locking element 51 each have, in the area of their free ends, latching elements in the form of three latching lugs 56, 56′, which will be discussed in more detail later. The same applies to a correspondingly designed latching structure, for example in the form of latching notches 57, which are provided on the guide rail 52.

The locking element 51 engages with its latching lugs 56, 56′ behind the cross-sectionally T-shaped guide rail 52 from both opposite sides. The latching lugs 56, 56′ engage in the latching notches 57 of the guide rail 52 in a fixing position, thereby preventing movement of the locking element 51 along the guide rail 52. By pressing the locking element 51 towards the foot part 3, the latching lugs 56, 56′ are disengaged from the latching notches 57, so that the locking element 51 takes up a displacement position. In the displacement position, the locking element 51 can be moved freely along the guide rail 52, so that the bending angle α between calf part 2 and foot part 3 can be adjusted or the patient is given a certain freedom of movement when walking.

A fixing element 512 in the form of a fixing screw protrudes through a through-hole centrally provided in the bulge 290. With its external thread, the fixing screw 512 is screwed into the internal thread of the through-opening 511 formed on the locking element 51. Via a washer 514, the fixing screw 512 also rests with its screw head in the area of the bulge 290 on the outside of the calf part 2. A spring element 53 in the form of a spiral spring, which is protruded by the fixing screw 512, is arranged between the locking element 51 and the washer 514. The spring element 53 applies a force to the locking element 51 directed towards the foot part 3 and thus holds the locking element 51 in its displacement position, i.e. out of engagement with the latching notches 57 of the guide rail 52, when the fixing screw 512 is only slightly screwed into the internal thread of the through-opening 511. By screwing the fixing element 512 into the internal thread of the through-opening 511, the locking element 51 can be brought into its fixing position. The locking element 51 is then pulled against the spring force caused by the spring element 53 in the direction of the calf part 2, bringing the latching lugs 56, 56′ into engagement with the latching notches 57 of the guide rail 52.

Although not shown here, it is also conceivable to provide the guide rail 52 as well as the locking element 51 without any latching structures and instead to provide only one stop surface each on the locking element as well as on the guide rail, which abut against each other in the fixing position. This would make it at least more difficult to move calf part 2 along the guide rail 52 of foot part 3 due to the frictional force caused by the fixing element 512. The locking element and the guide rail may, for example, have a friction-enhancing coating at the appropriate locations.

The second embodiment of a support shell assembly 1 shown in FIGS. 5 to 14 is similar, preferably identical, to the first embodiment shown in FIGS. 1 to 4, with a few exceptions which will be discussed in more detail below. In the following, mainly the differences between the second embodiment and the first embodiment will be explained.

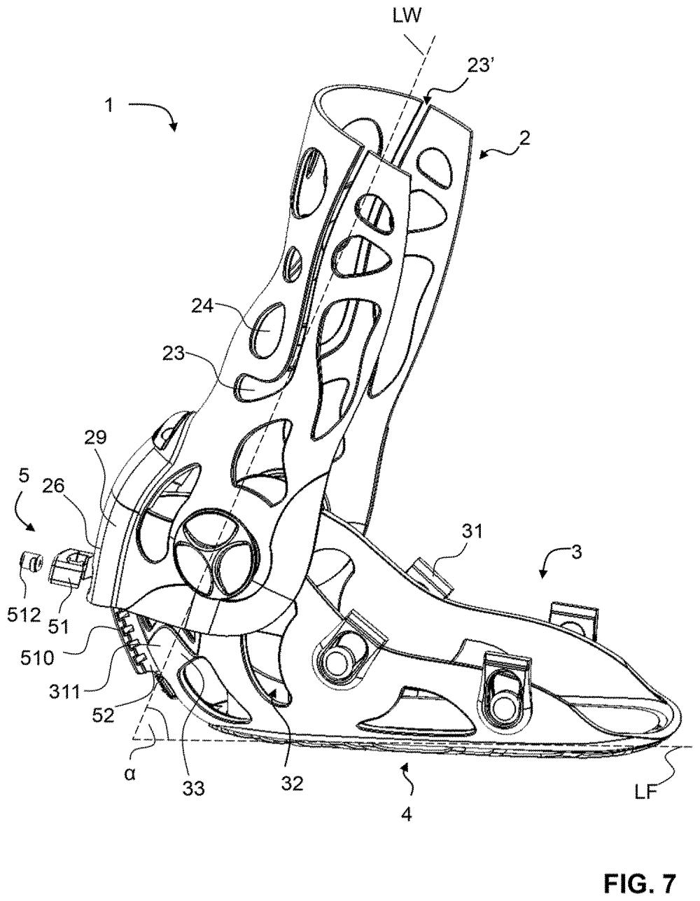

FIGS. 7 to 9 show that the locking element 51 has an essentially U-shaped outer shape, just as in the first embodiment, and has two legs 54, 54′ and a bar 55 connecting these two legs 54, 54′. In contrast to the first embodiment, however, the calf part 2 in turn has a recess 27 in the proximal region 26 at a lower end, which extends completely through the calf part 2 and the guide groove 25 formed there and is designed to receive the locking element 51 with a substantially precise fit. As a result, the locking element 51 accommodated in the recess 27 can be displaced along the guide rail 52 by a user, such as a doctor, together with the calf part 3.

In the area of the bar 55, the two legs 54, 54′ of the second embodiment each have an incision 58, 58′ into which the spring element 53, here in the form of a leaf spring, can be inserted. In the inserted state, the leaf spring 53 extends essentially along the entire length of the bar 55 and has a curvature 59, which is curved towards the guide rail 52 (the actual curvature is therefore preferably oriented in the opposite direction to the curvature shown incorrectly in FIGS. 8 and 9). This curvature 59 produces a spring force FK which acts on the locking element 51 and causes the locking element 51 to be movable between a displacement position and a fixing position, but is pressed towards the fixing position. In the fixing position, the spring force FK of the leaf spring 53 acts on the locking element 51 in such a way that a displacement of the locking element 51, and thus of the calf part 2, along the guide rail 52 is made more difficult or even impossible. The calf part 2 is thus pr-fixed to the guide rail 52 by means of the locking element 51. This fixing position is achieved because the curvature 59 of the leaf spring 53 comes to rest on a contact surface 510 of the guide rail 52 and causes the locking element 51 to be pressed outwards away from the contact surface 510 due to the spring force FK. In doing so, the latching lugs 56, 56′ of the locking element 51 come into engagement with the latching notches 57 of the guide rail 52 and displacement of the calf part 2 along the guide rail 52 is no longer possible.

In order to transfer the locking element 51 into the displacement position in which displacement of the locking element 51, and thus of calf part 2, is possible, a force GK opposing the spring force FK must be applied. That is to say, if a corresponding counterforce GK is applied to the spring element 53, in other words, if the locking element 51 is pressed from the outside through the recess 27 in the direction of the guide rail 52, the engagement between the latching lugs 56, 56′ of the locking element 51 and the latching notches 57 of the guide rail 52 is released. The locking element 51, and thus also the calf part 2, can then be moved along the guide rail 52. It is also decisive that this counterforce GK is not only opposite to the spring force FK of the spring element 53, but also has a strength which is equal to or greater than the strength of the spring force FK.

As also shown in FIGS. 8 and 9, the bar 55 of the locking element 51 can have a through-opening 511 through which a fixing element 512 can be inserted to additionally secure or fix the locking element 51 to the guide rail 52 in the fixing position. In particular, it is conceivable to provide the through-opening 511 with an internal thread and to use a fixing screw with an external thread as fixing element 512, which can then be screwed into the through-opening 511. This fixing screw 512 is then screwed in the direction of the guide rail 52 until the fixing screw 512 strikes the contact surface 510 of the guide rail 52. In the present case, the spring element 53 therefore also has a through-opening 513 through which the fixing screw 512 is screwed. However, it is also conceivable not to provide such a through-opening 513 on the spring element 53. In this case, the fixing screw 512 would not hit the contact surface 510 of the guide rail 52 directly when screwed in, but the fixing screw 512 would press the spring element 53 onto the contact surface 510. In both cases, the fixing screw 512 generates a force on the locking element 51, which presses the locking element 51 outwards away from the guide rail 510 in the same way as the spring element 53, whereby the latching lugs 56, 56′ of the locking element 51 are brought into maximum engagement with the latching notches 57 of the guide rail 52, respectively whereby a stop is created between the stop surface of the locking element and the guide rail 52. This prevents the locking element 51 from moving along the guide rail 52. This fixing force acting in addition to the spring force FK reduces the risk of unintentional displacement of the calf part 2 along the guide rail 52 in the fixing position, and thus the unintentional adjustment of a previously optimally set bending angle α between the foot part 3 and the calf part 2, thus increasing the wearing safety of the support shell assembly 1.

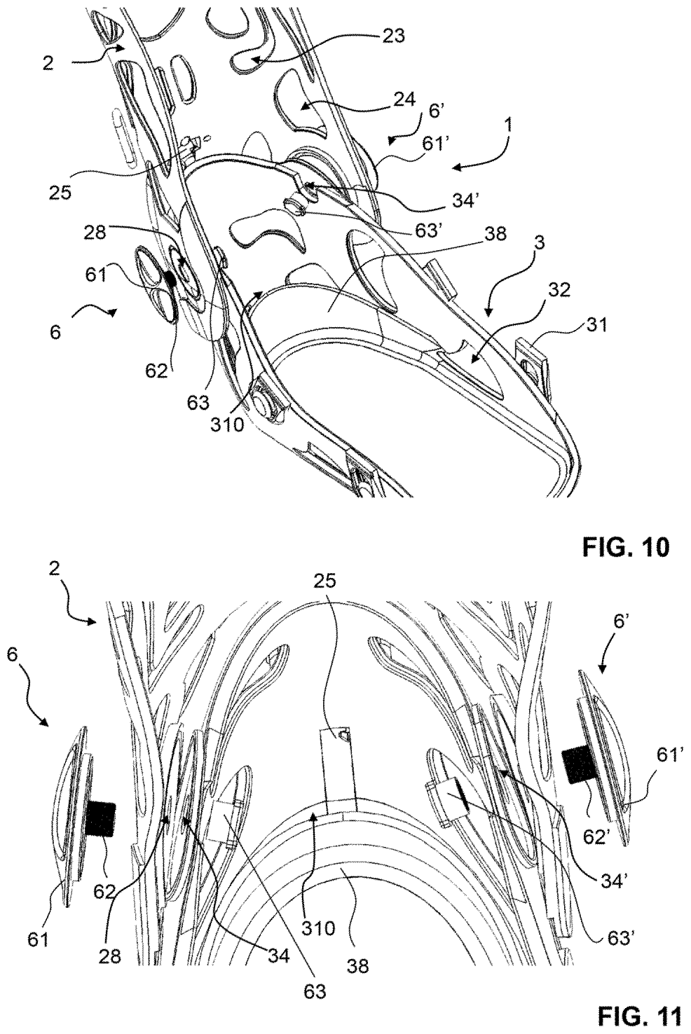

In particular from FIGS. 10 and 11 it can be seen that the support shell assembly 1 according to the second embodiment also includes an adjustment device 6, 6′ for the articulated connection between the calf part 2 and the foot part 3. The first embodiment shown in FIGS. 1 to 4 also has such an articulation device. This adjustment device 6, 6′ is arranged laterally on both sides of the support shell assembly 1 and comprises in each case a rotary knob 61, 61′ with a joint rod 62, 62′ and a journal 63, 63′. Both the calf part 2 and the foot part 3 each have an opening 28, 28′, 34, 34′ on both sides, through which the joint rod 62, 62′ of the rotary knob 61, 61′ extends from outside the support shell assembly 1 into the interior of the support shell assembly 1 and is received there non-rotatably in the journal 63, 63′. The joint rod 62, 62′ can, for example, have an external thread which is screwed into a corresponding internal thread formed in the journal 63, 63′. When the support shell assembly 1 is worn by a patient, the openings 28, 28′, 34, 34′ are positioned slightly offset behind the patient's ankle axis.

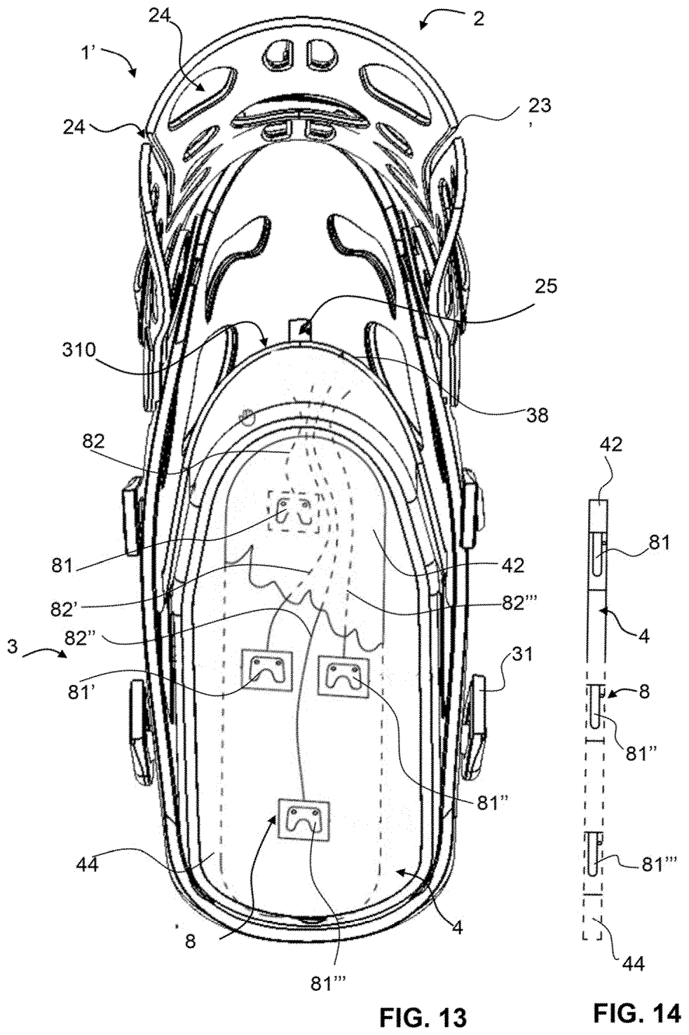

As mentioned above, the support shell assembly 1 of both the first and the second embodiment also has a sole part 4, which can be connected to the foot part 3. In particular, FIG. 12 shows that the sole part 4 can be detachably attached to foot part 3, for example by means of a snap-in or latching connection. For this purpose, the foot part 3 can have projections 35 which snap or latch into corresponding recesses 41 in the sole part 4 or vice versa. For a more stable connection of the sole part 4 with the foot part 3, the sole part 4 can additionally have an extended connecting structure 43 in a proximal area 42, which can be connected with a correspondingly designed connecting structure 36 in the proximal region 33 of the foot part 3. This connection can also be a snap-in or latching connection. This means that the sole part 4 can be very easily attached to the foot part 3 by “clicking it on”. However, instead of a removable sole part 4, the foot part 3 and the sole part 4 can also be inseparably connected to each other or formed as one piece. The sole part 4 can optionally have a pressure sensor device to detect the treading load on the sole part 4, as explained below in relation to the second embodiment shown in FIGS. 13 and 14.

In FIGS. 13 and 14 a sole part 4 is thus explained in relation to the second embodiment of the support shell assembly 1, which has a pressure sensor device 8 for detecting the treading load on the sole part 4. Of course, the first embodiment shown in FIGS. 1 to 4 can also have such a sole part 4 with a pressure sensor device.

In the example shown in FIG. 13 the pressure sensor device 8 has four pressure sensors 81, 81′, 81″, 81′″, 81″′, each of which is adapted to output a sensor signal. In this example, one pressure sensor 81 is located in the proximal area 42, one pressure sensor 81′″ in the distal area 44, and two pressure sensors 81′, 81″ are located next to each other in a central area of the sole part 4 located in between. While the proximally and distally located sensors 81, 81′″ primarily measure the treading load by the heel or the ball of the foot, the centrally located sensors 81′, 81″ serve to detect lateral tilting of the foot. In order that the sensor signals of the pressure sensors 81, 81′, 81″, 81′″ can be evaluated, each sensor is connected via its own line 82, 82′, 82″, 82′″ to an evaluation device (not shown). Via these lines 82, 82′, 82″, 82′″ the detected sensor signals are transmitted to the evaluation device and evaluated there into sensor values, in particular summed, thus enabling a comparison with predetermined reference values by the evaluation device. These reference values can correspond to a specified maximum value or maximum value range of the permissible treading load, which are stored in the evaluation device and are compared with the sensor values. Furthermore, an information transmission unit (also not shown) can be provided which is adapted to emit an optical and/or acoustic signal if the sensor values exceed the maximum value or the maximum value range. For example, the optical signal can be a flashing of a control lamp connected to the evaluation device and the acoustic signal can correspond to a whistling tone from a loudspeaker connected to the evaluation device. It is also conceivable to provide display means for indicating the determined sensor values, e.g. a display, or an energy supply device, e.g. in the form of a battery, for supplying the evaluation device and/or the information transmission unit with energy. One possibility is, for example, to embed the pressure sensors 81, 81′, 81″, 81′″ in an armour of sole part 4 and to connect each of them to an electronic circuit via the electrical line 82, 82′, 82″, 82′″. The electronic circuit may include a battery, e.g. a CR2032 button cell, a push button for setting the maximum value of the treading load, e.g. 5, 10, 15, 20, 25, 30, 35, 40 kg, an LED lamp which signals confirmation of the treading load by flashing, and a reset in the form of a calibration button. Furthermore, a loudspeaker, which emits an acoustic signal when the set maximum value is exceeded, as well as a display for indicating the treading load, can also be connected to the electronic circuit via further electronic lines. It is also conceivable to provide a work switch on the electronic circuit, which is adapted to switch on the energy supply in case of a treading load and to switch off the energy supply in case of no treading load. In other words, this switch fulfils a wake-up function to a certain extent, whereby a treading load is only determined if a patient wears the support assembly 1 and steps on the sole part 4. So that these elements do not impair a patient when wearing the support shell assembly 1, a receptacle 310 is provided in the support shell assembly 1, in which the evaluation device and/or the information transmission unit and/or the display means and/or the energy supply device can be accommodated. As can be seen in particular in FIGS. 6, 10 and 13, the foot part 3 can have a heel strap 38 for this purpose on the heel side, which is arranged in the foot part 3 at a distance from the proximal end 37 of the foot part 3 and forms a stop 39 for the heel of a patient in the distal direction. In the proximal direction, a receptacle is thus formed in the form of an interspace or gap 310 in which the elements connected to the sensors 81, 81′, 81″, 81″ can be accommodated and stowed away.

FIG. 15 shows an inlay 7 in the form of an inner shoe for one of the support shell assemblies 1 as described above. This inlay 7 is used for pre- or post-operative or post-traumatic immobilisation and stabilisation, e.g. for foot or ankle fractures, fractures of the lower leg, sprains, Achilles tendonitis, soft tissue injuries, athrodesis, cacaneus fractures, metatarsal fractures, etc. This is a comfortable, easy-to-fit inlay 7 for the patient, preferably made of a textile, which is an alternative treatment to plaster casts. For this purpose the inlay 7 comprises at least a first chamber 71 with a first valve device 73 and a second chamber 72 with a second valve device 74. The first chamber 71 is located in a heel-side or proximal area 75 of the inlay 7 and extends laterally at least partially along a foot-side area 76 of the inlay 7. The second chamber 72 is located in the foot-side area 76 of the inlay 7 and is framed both proximally and laterally by the first chamber 71. In other words, the second chamber 72 is arranged in a recess of the first chamber 71, with a space between the two chambers 71, 72 being formed here. The first chamber 71 corresponds to a cavity into which a fluid can be introduced via the first valve device 73. This results in a stiffening of the inlay 7 in the area of the first chamber 71. The fluid is preferably air, which is pumped into the first chamber 71, while the inlay 7 is arranged in a support shell assembly 1 and a patient wears the support shell assembly 1. The second chamber 72 is also a cavity, but contains a large number of moulded bodies 77 such as for example polystyrene balls. Unlike the first chamber 71, however, no fluid is filled into the second chamber 72 for stiffening. Instead, the stiffening of the inlay 7 in the area of the second chamber 72 is is achieved by discharging a fluid from the second chamber 72 via the second valve device 74. For example, a vacuum pump can be connected to the second valve assembly 74, which sucks the air out of the second chamber 72. Here, too, the patient wears the support shell assembly 1 with inlay 7, so that the second chamber 72 has the illustrated shape of the body part after evacuation. As can be seen from the schematic illustration in FIG. 16, a renewed deformability of the second chamber 72 can be achieved by filling a fluid such as air into the second chamber 72. This allows the moulded bodies 77 of the second chamber 72 to move and displace and thus re-adapt to the body part to be supported. When the air is then sucked out of the second chamber 72 again, the moulded bodies 77 remain in their newly adjusted position. The second chamber 72 can thus be reversibly stiffened. The same applies, of course, to the first chamber 71, from which the injected air can be released and then refilled. Thus, the inlay 7 enables a flexible, precisely fitting and secure embedding of the foot, ankle or calf, thus avoiding pressure points and promoting the reduction of pain and swelling.

In order to allow air to be introduced into or discharged from chambers 71, 72, the chambers must be made of a gas-tight material.

A flexible adjustment of the inlay 7 can be further achieved by integrating one or more pumps 78 into the inlay 7. For example, the inlay 7 can have a holder 79 for an air pump 78 for inflating the first chamber 71, which has the shape of a side pocket 79 and is sewn into the inlay 7, for example. The air pump 78 can be a manually operated hand pump which is then inserted into this side pocket 79.

It is advantageous to have a single pump with a single bellows, which can be switched from pump to suction mode and vice versa. Together with the first and the second valve device 73, 74 this pump then forms a pumping device which, depending on the mode of the pump, serves to inflate the first chamber 71 or to suck the fluid from the second chamber 72. The first valve device 73 and the second valve device 74 are then respectively arranged in close proximity to each other or coupled to the common pump via corresponding conduits.

The inner shoe has advantageously an outer layer made of a polyester fabric and an inner layer made of a terry cloth. Between the outer and the inner layer a foam is preferably arranged. The chambers 71 or 72 are advantageously located between the foam and the inner layer.

In addition or as an alternative to the inner shoe, a spacer fabric can also be provided on the inside with respect to the support shell assembly and the inner shoe, if any, which can serve to relieve pressure points, improve air circulation and/or absorb secretions.

LIST OF REFERENCE SIGNS

| | | | |
|---|---|---|---|
| 1 | Support shell assembly | 3 | Foot part |
| | | 31 | Belt straps |
| 2 | Calf part | 32 | Openings |
| 21 | Belt straps | 33 | Proximal region |
| 22 | Fastening belt | 34, 34′ | Opening |
| 23 | Slit | 35 | Projections |
| 24 | Openings | 36 | Connection structure |
| 25 | Guide groove | 37 | Proximal end |
| 26 | Proximal region | 38 | Heel strap |
| 27 | Recess | 39 | Stop |
| 28, 28′ | Opening | 310 | Gap |
| 29 | Curved region | 311 | Curved region |
| 290 | Bulge | | |
| | | 4 | Sole part |
| 41 | Recesses | 7 | Inner shoe |
| 42 | Proximal area | 71 | First Chamber |
| 43 | Extended connecting | 72 | Second Chamber |
| | structure | | |
| 44 | Distal area | 73 | First valve device |
| | | 74 | Second valve device |
| 5 | Locking device | 75 | Proximal area |
| 51 | Locking element | 76 | Foot side area |
| 52 | Guide rail | 77 | Moulded bodies |
| 53 | Spring element | 78 | Pump |
| 54, 54′ | Leg | 79 | Side pocket |
| 55 | Base | | |
| 56, 56′ | Latching elements | 8 | Pressure sensor device |
| 57 | Latching notches | 81, 81′, | Pressure sensors |
| | | 81′, 81′″ | |
| 58, 58′ | Incision | 82, 82′, | Lines |
| | | 82″, 82′″ | |
| 59 | Curvature | | |
| 510 | Contact surface | 91 | Tibia part |
| 511 | Through-opening | 92 | Foot back part |
| 512 | Fixing element | | |
| 513 | Through-opening | FK | Spring force |
| 514 | Washer | GK | Counterforce |
| | | α | Bending angle |
| 6, 6′ | Adjustment device | LW | Main longitudinal axis |
| 61, 61′ | Rotary knob | | of calf part |
| 62, 62′ | Joint rod | FW | Main longitudinal axis |
| 63, 63′ | Journal | | of foot part |

1. A support shell assembly comprising:
a calf part;
a foot part which is pivotably connectable to the calf part; and
a locking device with a guide rail attached to the calf part or to the foot part and with a locking element,
wherein the locking element, for adjusting a bending angle between the calf part and the foot part, is displaceable along the guide rail and fixable in a desired position on the guide rail, and
wherein the locking device additionally comprises a spring element which applies a spring force to the locking element in such a way that displacement of the locking element along the guide rail is facilitated or made more difficult.

2. The support shell assembly according to claim 1, wherein the guide rail has a latching structure and the locking element has one or more latching elements, and wherein the locking element is movable between a displacement position, in which the latching element or elements are out of engagement with the latching structure, and a fixing position in which the latching element or elements are in engagement with the latching structure.

3. The support shell assembly according to claim 2, wherein the locking element is acted upon by the spring element with a spring force in the direction of the displacement position.

4. The support shell assembly according to claim 2, wherein the locking element is acted upon by the spring element with a spring force in the direction of the fixing position.

5. The support shell assembly according to claim 1, wherein the locking device further has at least one fixing element for fixing the locking element in the desired position on the guide rail.

6. The support shell assembly according to claim 1, wherein the foot part is curved on the heel side and is movable with this curved region at least partially along a correspondingly curved region of the calf part.

7. The support shell assembly according to claim 1, wherein the guide rail of the locking device is arranged on the heel side of the foot part, or
wherein the guide rail of the locking device is arranged on the heel side of the calf part.

8. The support shell assembly according to claim 1, wherein the locking element is arranged between the calf part and the foot part, and wherein the locking element is acted upon by the spring element with a spring force in the direction of the foot part or the calf part.

9. The support shell assembly according to claim 1, wherein the guide rail is arranged on the outside of the foot part and the calf part has a guide groove on the inside for guided reception of the guide rail.

10. The support shell assembly according to claim 1, wherein the calf part has at least one slit which extends at least partially into the calf part so that a flexible adaptation of the calf part to the calf of a patient is enabled.

11. The support shell assembly according to claim 1, further comprising a sole part which can be connected to the foot part, wherein the sole part has a pressure sensor device for detecting the treading load of the sole part by a patient, and
wherein the pressure sensor device comprises at least one pressure sensor adapted to output a sensor signal.

12. The support shell assembly according to claim 11, further comprising:
an evaluation device in which a predetermined maximum value and/or maximum value range of the permissible treading load can be stored, and which is adapted to determine a sensor value from the sensor signal of the pressure sensor and to compare the determined sensor value with the predetermined maximum value and/or the maximum value range, and
an information transmission unit which is adapted to emit an optical and/or acoustic signal if the maximum value and/or the maximum value range is exceeded by the determined sensor value.

13. The support shell assembly according to claim 12, further comprising;
an energy supply device for supplying the evaluation device and/or the information transmission unit with energy, and a work switch, wherein the work switch is adapted to switch on the energy supply in the event of a treading load and to switch off the energy supply if no treading load is detected for a certain time period.

14. The support shell assembly according to claim 1, further comprising a tibia part adapted to wrap around the patient's lower leg together with the calf part.

15. An inlay for a support shell assembly comprising:
a first chamber;
a second chamber which contains a plurality of moulded bodies,
a first valve device by means of which a fluid can be introduced into the first chamber to stiffen the inlay in a first area; and
a second valve device by means of which a fluid can be discharged from the second chamber in order to stiffen the inlay in a second area.

16. The inlay according to claim 15, wherein the first valve device and the second valve device are formed by a common pumping device which is switchable from a pumping mode to a suction mode and vice versa.

17. The support shell assembly according to claim 5, wherein the at least one fixing element is in the form of a fixing screw.

18. The support shell assembly according to claim 7, wherein the guide rail of the locking device is arranged on the heel side of the foot part and is formed in one piece with the foot part.

19. The support shell assembly according to claim 7, wherein the guide rail of the locking device is arranged on the heel side of the calf part and is formed in one piece with the calf part.

20. The inlay according to claim 15, wherein the inlay is an inner shoe.
